(19) United States
(12) Patent Application Publication
(10) Pub. No.: US 2016/0052962 A1
(43) Pub. Date: Feb. 25, 2016

(54) METHOD FOR ISOLATING COLLAGEN FROM JELLYFISH BY USING RADIATION

(71) Applicant: KOREA ATOMIC ENERGY RESEARCH INSTITUTE, Daejeon (KR)

(72) Inventors: Youn-Mook LIM, Jeollabuk-do (KR); Sung In JEONG, Chungcheongbuk-do (KR); Hui-Jeong GWON, Jeollabuk-do (KR); Jong Seok PARK, Jeollabuk-do (KR); Young-Chang NHO, Daejeon (KR); Phil-Hyun KANG, Daejeon (KR); Young-Jin KIM, Daejeon (KR)

(21) Appl. No.: 14/863,692

(22) Filed: Sep. 24, 2015

Related U.S. Application Data
(63) Continuation of application No. PCT/KR2014/001986, filed on Mar. 11, 2014.

(30) Foreign Application Priority Data
Mar. 29, 2013 (KR) 10-2013-0034745
Mar. 11, 2014 (KR) 10-2014-0028149

(51) Int. Cl. C07K 1/36; C07K 14/435; C07K 14/78

(52) U.S. Cl. CPC C07K 1/36; C07K 14/78; C07K 14/43595

(57) ABSTRACT

The present invention relates to a method for separating collagen from jellyfish by using radiation. More precisely, acid-soluble collagen and attelo collagen were prepared in this invention by using the method combining irradiation technique and chemical treatment. This method of the invention is expected to be useful for the separation of collagen from jellyfish with low costs but high yield.

### BACKGROUND OF THE INVENTION

1. Field of the Invention

The present invention relates to a method for extracting collagen from jellyfish by using a irradiation technique. More precisely, the invention relates to a method for separating collagen from jellyfish with low costs but high yield by using the method combining irradiation technique and chemical treatment.

2. Description of the Related Art

Collagen is a major component of extracellular matrix, which is distributed in the skin, bone, and cartilage protein. Collagen is a fibrous high-molecular protein having the structure of triple helix. The diameter of collagen is about 14˜15 Å and the length is 2800 Å. The molecular weight of collagen is approximately 300,000 Da. The physical and biological stability of the collagen structure is resulted from the cross-linking between tropocollagen molecules which are the basic molecules of fibrous protein. Generally the peptide structure of collagen is composed of (Gly-X—Y)n, wherein X is proline and Y is hydroxyproline that fills up the ⅓ of the structure and the remaining ⅔ of the structure is filled with other amino acids.

Collagen is a functional material that is widely used in industry including the field of food, medicine, cosmetics, and cell culture, etc. In the food industry, collagen is used as an edible casing or carrier or an additive to increase taste of food such as sausage or ham. Recently the demand of collagen is increasing because of its functionality working in fixing cell adhesion, inducing cell division and differentiation, inducing thrombolysis, increasing memory power, wound healing, and protecting gastric mucosa, etc. The animal origin collagen is most used as a medicinal material. However, because of the high risk of such animal origin collagen of exposing on infectious pathogens (bovine spongiform encephalopathy, avian influenza, transmissible spongiform encephalopathy, etc), human collagen has been tried to reduce such risk. But, human collagen is still limited and has problems of low productivity resulted from the difficulty in extraction and high processing costs along with ethical and social issues. To overcome the said problems, it is actively attempted to develop and commercialize marine organism originated biopolymer for the preparation of wound-covering material, drug delivery material, and artificial organ material for regenerative medicine which seem to be free from cytotoxicity and side effect of immune reaction but to have high cell compatibility compared with animal origin protein. In Korea, companies give weight to the separation and purification process of high purity biopolymer for medicine and to the development of degradation process. The trial product is at a developmental stage but it is still required to establish a novel technique to increase price competitiveness.

Studies on the marine organism originated collagen have been conducted with the acid-soluble collagen extracted from jellyfish and fish skin and bone of adult and juvenile fish. The marine organism originated collagen was compared with the animal origin collagen in amino acid composition, denaturation temperature, and solubility, etc. As a result, the marine organism originated collagen was confirmed to have the similar structure with the animal origin collagen. Particularly, jellyfish collagen is confirmed to be effective in increasing skin elasticity, in regulating blood circulation, and in the treatment of arthritis, hypertension, bronchitis, and asthma. Besides, the jellyfish collagen has a high potential for the industrial use in the fields of high protein diet food, cosmetics, and medicine.

In the meantime, mass propagation of jellyfish resulted from global warming has a bad effect on ecosystem and the elimination of excessive jellyfish is also a problem. Jellyfish has been limited in use as a simple processed food so far. To extract collagen from jellyfish, the conventional method depends on the simple chemical treatment with acid, alkali, and salt. The method depending on the chemical treatment, however, has problems of accompanying environmental pollution and low yield that is a disadvantage for commercialization.

To overcome the above problems, the present inventors have focused on the development of a novel, more efficient method for separating collagen from jellyfish. As a result, the inventors confirmed that a method combining irradiation technique and chemical treatment on jellyfish could be advantageous in reducing costs but increasing yield and efficiency in collagen separation, leading to the completion of the present invention.

### SUMMARY OF THE INVENTION

It is an object of the present invention to provide a method for separating acid-soluble collagen from jellyfish.

It is another object of the present invention to provide a method for preparing attelo collagen containing the step of treating the acid-soluble collagen prepared by the above method with protease and drying the resultant product.

To achieve the above objects, the present invention provides a method for separating acid-soluble collagen from jellyfish comprising the following steps:

1) washing and pulverizing jellyfish;

2) dipping the pulverized jellyfish prepared in step 1) in an acid solution;

3) irradiating the solution of step 2), followed by stirring; and

4) filtering the stirred solution of step 3) and drying thereof.

The present invention also provides a method for preparing attelo collagen containing the step of treating the acid-soluble collagen prepared by the above method with protease and drying the resultant product.

### Advantageous Effect

The method combining irradiation technique and chemical treatment on jellyfish is advantageous in producing collagen with low costs but high yield. Compared with the conventional method depending on the chemical treatment only, the method of the present invention reduces the costs but increases yield in addition to prevent environmental pollution with brining the effect of eliminating harmful excessive jellyfish. Further, the method of the invention can be efficiently used as a separation technique usable for the preparation of jellyfish collagen raw material and biomaterial, which is a basic technique required for the field of tissue engineering.

### BRIEF DESCRIPTION OF THE DRAWINGS

The application of the preferred embodiments of the present invention is best understood with reference to the accompanying drawings, wherein:

FIG. 1 is a flow chart illustrating the method for separating collagen from jellyfish of the present invention.

FIG. 2 is a diagram illustrating the collagen extracted from jellyfish according to the dosage of gamma-ray irradiation.

FIG. 3 is a diagram illustrating the weight changes of washed jellyfish.

FIG. 4 is a diagram illustrating the particle size of jellyfish according to the grinding time.

FIG. 5 is a diagram illustrating the yield of the acid-soluble collagen extracted from jellyfish according to the stirring time after the gamma-ray irradiation.

FIG. 6 is a diagram illustrating the yield of the acid-soluble collagen extracted from jellyfish according to the dosage of gamma-ray irradiation.

FIG. 7 is a diagram illustrating the extraction rate of the attelo collagen prepared from jellyfish according to the dosage of gamma-ray irradiation.

FIG. 8 is a diagram illustrating the chemical characteristics of the acid-soluble collagen extracted from jellyfish according to the dosage of gamma-ray irradiation.

FIG. 9 is a diagram illustrating the thermal characteristics of the attelo collagen prepared from jellyfish according to the dosage of gamma-ray irradiation.

FIG. 10 is a diagram illustrating the components of the attelo collagen prepared from jellyfish according to the dosage of gamma-ray irradiation.

### DESCRIPTION OF THE PREFERRED EMBODIMENTS

Hereinafter, the present invention is described in detail.

The present invention provides a method for separating acid-soluble collagen from jellyfish comprising the following steps:

1) washing and pulverizing jellyfish;

2) dipping the pulverized jellyfish prepared in step 1) in an acid solution;

3) irradiating the solution of step 2), followed by stirring; and

4) filtering the stirred solution of step 3) and drying thereof.

In the method of the invention, step 1) is to wash and pulverize jellyfish.

In the method of the invention, the washing and pulverizing jellyfish in step 1) is preferably performed by the following steps, but not always limited thereto:

I) pulverizing the washed jellyfish;

II) freeze-drying the pulverized jellyfish of step I); and

III) pulverizing the freeze-dried jellyfish of step II).

The freeze-dried jellyfish is preferably pulverized in the particle size of 100˜3000 μm, but not always limited thereto.

In the method of the invention, step 2) is to dip the pulverized jellyfish in an acid solution.

The acid solution herein is preferably selected from the group consisting of acetic acid solution, citric acid solution, and formic acid solution, and is more preferably acetic acid solution, but not always limited thereto.

The concentration of the acid solution herein is 0.01 M˜2.0 M, preferably 0.1 M˜1.5 M, more preferably 0.3 M˜1.0 M, and most preferably 0.5 M, but not always limited thereto.

In the method of the invention, step 3) is to irradiate the solution and to stir thereof.

The radiation used herein is preferably gamma-ray or electron beam, and more preferably gamma-ray, but not always limited thereto.

The dosage of irradiation herein is 5 kGy˜200 kGy, preferably 5 kGy˜100 kGy, more preferably 5 kGy˜50 kGy, more preferably 5 kGy˜25 kGy, and most preferably 10 kGy, but not always limited thereto.

If the radiation dose is out of the above range, for example the radiation dose under 5 kGy would bring no effect on collagen extraction by irradiation and if the radiation dose is over 200 kGy collagen would be decomposed or denatured.

In the method of the invention, step 4) is to filter the stirred solution and to dry thereof according to the following steps, but not always limited thereto:

i) obtaining a precipitate from the filtrate remaining after filtering the stirred solution;

ii) obtaining the supernatant after dissolving the precipitate of step i) in an acid solution;

iii) obtaining a precipitate by adding salt to the supernatant of step ii); and

iv) dissolving the precipitate of step iii) in an acid solution, followed by dilution and freeze-drying.

The present invention also provides a method for preparing attelo collagen containing the step of treating the acid-soluble collagen prepared by the above method with protease and drying the resultant product.

The protease herein is preferably pepsin or trypsin, and more preferably pepsin, but not always limited thereto.

The protease concentration is preferably 1˜10 (w/w) %, and more preferably 3˜6 (w/w) %, and most preferably 5 (w/w) %, but not always limited thereto.

The protease is used to eliminate Telo peptide of collagen. The elimination of helix structure in the end of collagen molecule results in the elimination of antigenicity, suggesting that the collagen molecule can be easily used as a biomolecule.

The drying herein is performed by quick freezing at −178˜−70° C., but not always limited thereto.

The present invention also provides a specific method for preparing attelo collagen from jellyfish comprising the following steps:

a) dissolving the acid-soluble collagen in the acid/pepsin mixed solution, followed by stirring;

b) dissolving the precipitate obtained from the stirred mixture of step a) in acid, to which salt is added to precipitate collagen; and

c) dissolving the precipitated collagen of step b) in acid, followed by dilution and freezing.

In the present invention, the method for separating collagen from jellyfish by using radiation is characterized by the steps of washing jellyfish and pulverizing thereof; dipping the pulverized jellyfish in an acid solution, followed by irradiation; extracting the acid-soluble collagen; and treating the extracted collagen with pepsin, followed by freeze-drying. By this method, attelo collagen can be successfully prepared.

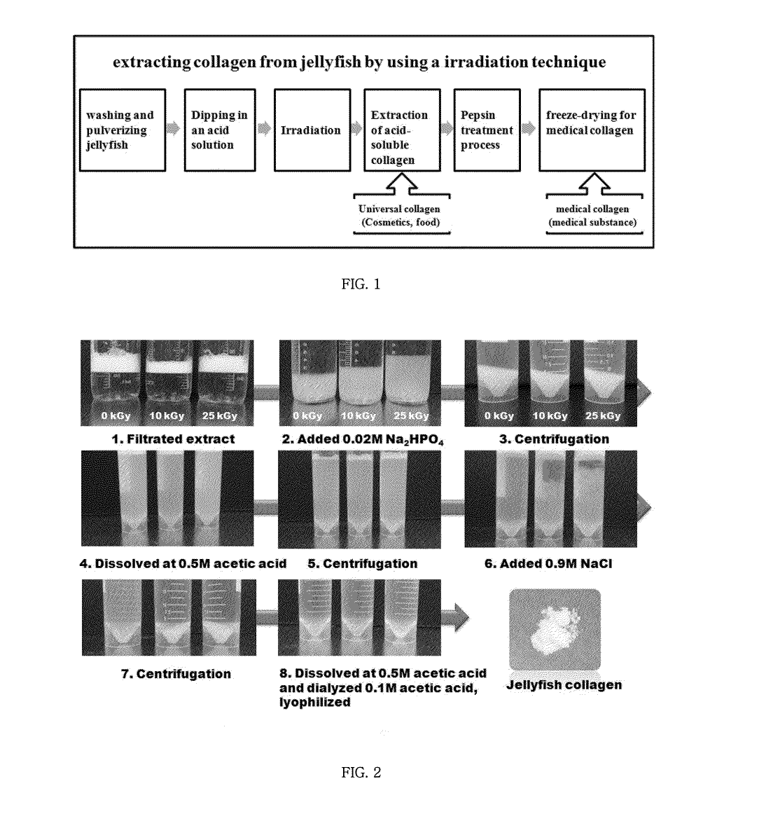

Particularly, as shown in the schematic diagrams of FIG. 1 and FIG. 2, jellyfish was washed and pulverized first. The pulverized jellyfish was dipped in an acid solution, which was irradiated and stirred. The stirred solution was filtered to obtain a precipitate. The obtained precipitate was dissolved in acid. Supernatant was obtained therefrom, to which salt was added to obtain a precipitate. The precipitate was dissolved in acid, followed by dilution and freeze-drying to extract acid-soluble collagen. The acid-soluble collagen was dissolved a mixed solution of acid and pepsin. The mixture was stirred and a precipitate was obtained from the stirred solution again. The precipitate was dissolved in acid, to which salt was added to precipitate collagen. The collagen was dissolved in acid and diluted, followed by freeze-drying. As a result, attelo collagen was prepared.

In a preferred embodiment of the present invention, jellyfish (Nemopilema nomuri Kishinouye) was washed with distilled water, followed by pulverization. The pulverized jellyfish was dipped in acetic acid, followed by irradiation with gamma-ray. The irradiated acid solution containing the jellyfish was stirred and filtered. The filtrate was diluted and a precipitate was obtained. The precipitate was dissolved in acetic acid and supernatant was obtained therefrom. Sodium chloride was added to the supernatant, and a precipitate was obtained therefrom. The precipitate was dissolved again in acetic acid, followed by freeze-drying. As a result, acid-soluble collagen was obtained. The acid-soluble collagen was dissolved in pepsin/acetic acid, followed by stirring. A precipitate was obtained from the stirred solution, which was dissolved in acid. Salt was added thereto to precipitate collagen. The precipitated collagen was dissolved in acid, diluted, and freeze-dried. As a result, attelo collagen was prepared (see FIGS. 1 and 2).

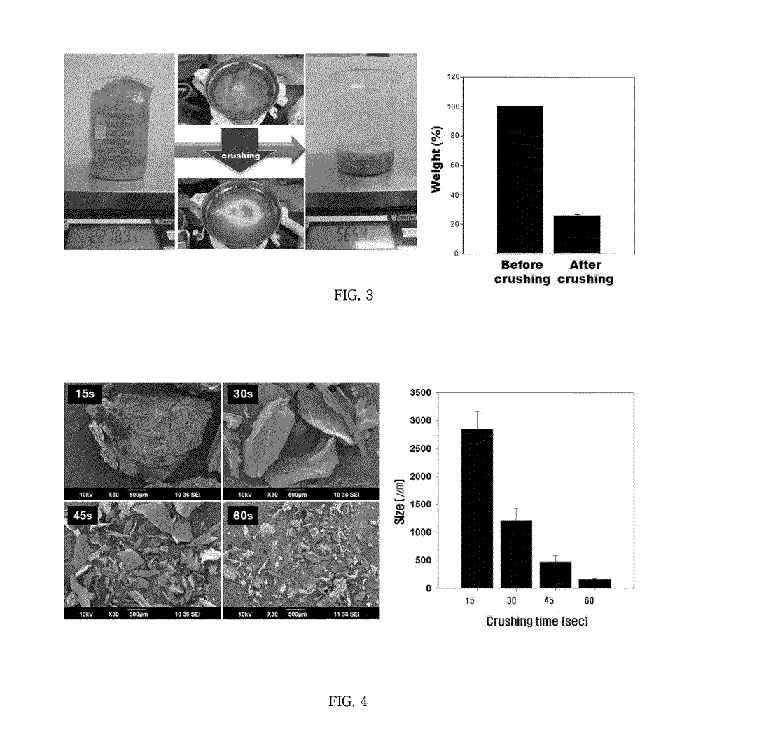

The weight and the particle size of the pulverized jellyfish were investigated. When jellyfish was pulverized before freeze-drying, the weight was reduced by 25%, and the longer pulverizing was taking the smaller particle size of the freeze-dried jellyfish. In particular, when jellyfish was pulverized for 60 seconds, the particle size thereof was about 128 μm (see FIGS. 3 and 4, and Tables 1 and 2).

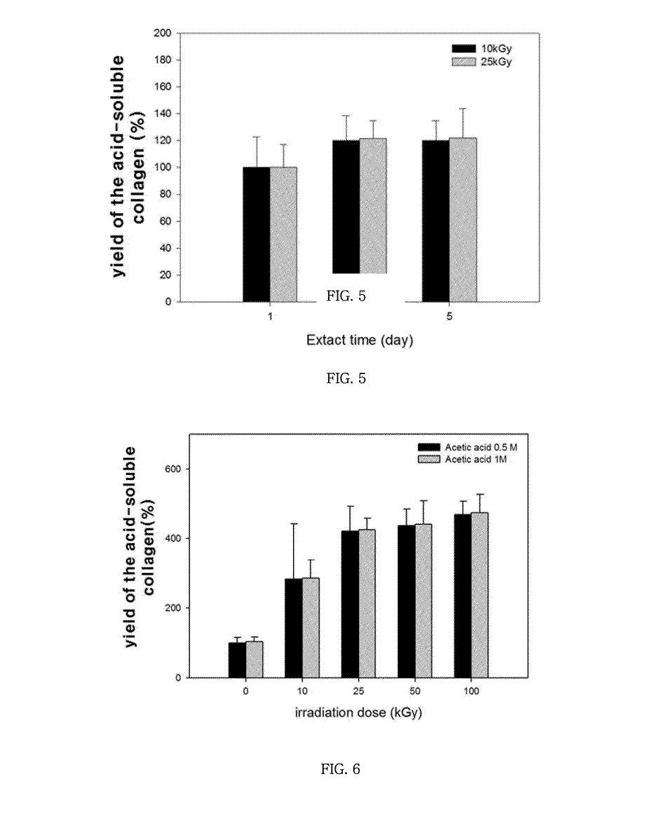

In an experimental example of the invention, the extraction time dependent acid-soluble collagen extraction was investigated. When collagen was extracted after stirring the reaction mixture for 3 or days, the extraction yield was significantly increased (see FIG. 5).

In an experimental example of the invention, the radiation dose dependent acid-soluble collagen extraction was investigated. As the radiation dose was increased, the collagen yield was increased, and particularly at the radiation doses of 10 kGy and 25 kGy, the collagen yield was significantly increased. However, at the radiation dose of 100 kGy, the color of collagen was changed to light-yellow (see FIG. 6).

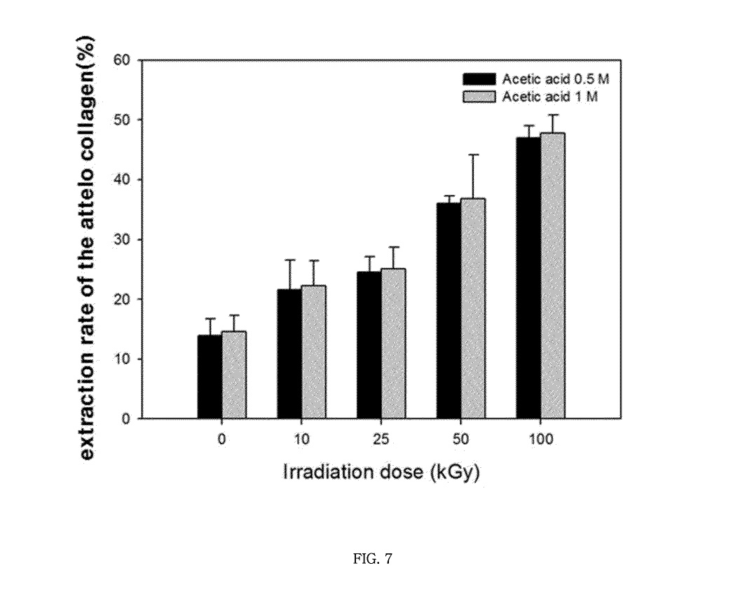

In an experimental example of the invention, the radiation dose dependent attelo collagen extraction was investigated. As a result, as the radiation dose was increased, the weight change of attelo collagen was less (see FIG. 7).

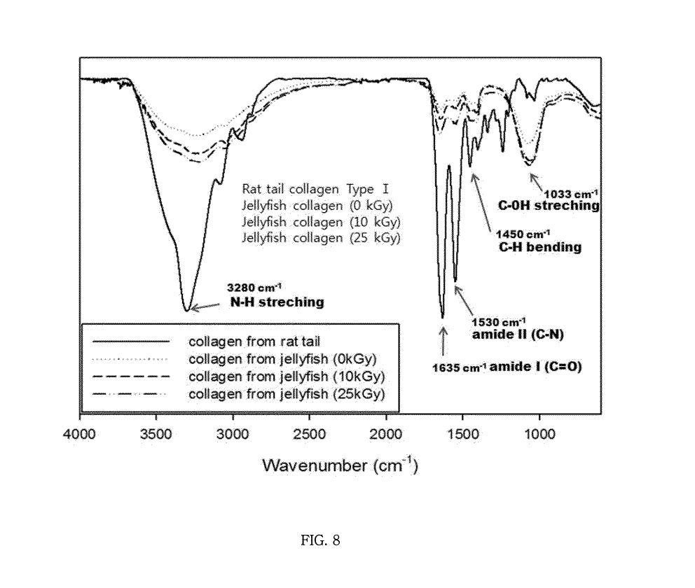

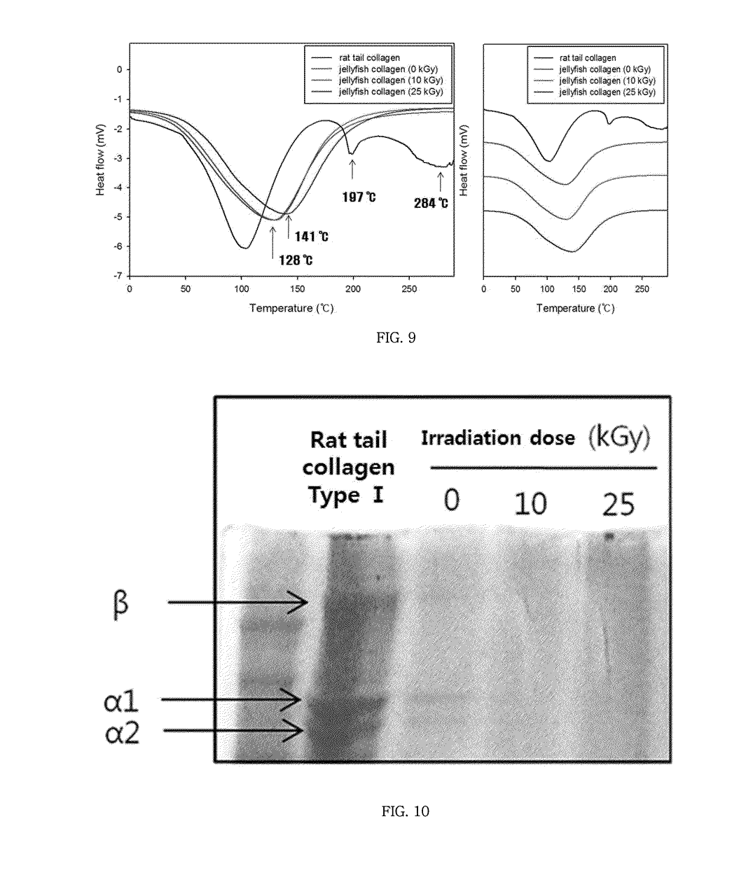

In an experimental example of the invention, the radiation dose dependent chemical and thermal characteristics of collagen were investigated. As a result, the collagen extracted from the irradiated jellyfish according to the present invention was confirmed to have animal collagen like spectrum pattern, indicating that the irradiation did not affect the chemical or thermal characteristics of collagen (see FIG. 8 and FIG. 9).

In an experimental example of the invention, the radiation dose dependent attelo collagen composition changes were investigated. The attelo collagen extracted from the jellyfish irradiated with gamma-ray at the dosage of 10 kGy demonstrated animal collagen like composition (see FIG. 10 and Table 3).

Therefore, the method for separating acid-soluble collagen from jellyfish and the method for preparing attelo collagen of the present invention have advantages of high collagen yield, compared with the conventional method depending on chemical treatment, and saving costs owing to the cut out of chemicals, and preventing environmental pollution because they can use excessive jellyfish that do harm on ecosystem.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Washing and Pulverizing Jellyfish

Nemopilema nomuri Kishinouye was distributed from National Fisheries Research & Development Institute, Korea and transported in an ice box filled with ice from Biheung Port (Gunsan, Korea). The salted jellyfish was washed with 4° C. distilled water for 3 days. The washed jellyfish was pulverized in a mixer, and the moisture was eliminated by filtering with a filter net. The jellyfish was then freeze-dried, which was pulverized in a mixer.

### Example 2

### Dipping Jellyfish in an Acid Solution and Irradiation

The pulverized jellyfish prepared in Example 1 was dipped in 0.5 M acetic acid (glacial grade, Merck, Darmstadt, Germany), which was irradiated with gamma-ray (⁶⁰Co, Pencil type, MDS Nordion, Canada) at the dosage of 10 kGy/hr, total 10˜100 kGy, followed by stirring at 4° C. for 2 weeks.

### Example 3

### Extraction of Acid-Soluble Collagen from Jellyfish

The stirred solution prepared in Example 2 was filtered and the filtrate was diluted with 0.02 M Na₂HPO₄ (Sigma, St. Luis Mo., USA) at the ratio of 1:3 (v/v), followed by dialysis. A precipitate was obtained therefrom by centrifugation (2000 rpm, 6 min). The precipitate was dissolved in 0.5 M acetic acid, followed by centrifugation (2000 rpm, 6 min) to obtain supernatant. Sodium chloride (NaCl, Sigma) was added to the supernatant at the concentration of 0.9 M and the obtained precipitate was dissolved in 0.5 M acetic acid. The solution was diluted until the acetic acid concentration reached 0.1 M, followed by freeze-drying. As a result, acid-soluble collagen was obtained.

### Example 4

### Preparation of Attelo Collagen

The acid-soluble collagen separated in Example 3 was dissolved in the mixed solution containing 0.5 M acetic acid and 5 w/w % pepsin (EC 3.4.23.1, 2× crystallized, Tokyo chemical industry, Japan), followed by stirring at 4° C. for 24 hours. The stirred solution was diluted with 0.02 disodium hydrogen phosphate (Na₂HPO₄). A precipitate was obtained therefrom by centrifugation. The precipitate was dissolved in 0.5 M acetic acid. Sodium chloride was added thereto at the concentration of 0.9 M in order to precipitate collagen. The precipitated collagen was dissolved in 0.5 M acetic acid again, and diluted until the acetic acid concentration reached 0.1 M, followed by freeze-drying. As a result, attelo collagen was prepared.

### Experimental Example 1

### Investigation of the Jellyfish Weight According to Pulverization

Nemopilema nomuri Kishinouye is a giant jellyfish, 90% of which is composed of water. Therefore, the volume of jellyfish needed to be reduced before being freeze-dried. To investigate the weight of jellyfish over pulverization, the jellyfish washed by the same manner as described in Example 1 was pulverized in a mixer. Then, the weight was measured.

As a result, as shown in FIG. 3 and Table 1, the weight of jellyfish was reduced by 25% after the pulverization (FIG. 3 and Table 1).

TABLE 1

| | Weight loss after |
|---|---|
| | pulverization (%) |
| Average weight loss | 25.50138 |
| Standard error | 1.101062 |

### Experimental Example 2

### Investigation of the Particle Size of Jellyfish According to the Pulverization Time

To investigate the particle size of jellyfish over the pulverization time, the freeze-dried jellyfish prepared by the method described in Example 1 was pulverized for 0, 15, 30, 45, and 60 seconds, followed by observation under electron microscope to measure the particle size of the pulverized jellyfish.

As a result, as shown in FIG. 4 and Table 2, the particle size of jellyfish became smaller over the pulverization time and particularly when the freeze-dried jellyfish was pulverized for 60 seconds, the particle size became 17 times smaller than the particle size resulted from pulverizing for 15 seconds (FIG. 4 and Table 2).

TABLE 2

Pulverization time (sec)

| | 15 | 30 | 45 | 60 |
|---|---|---|---|---|
| Average particle | 2841.98 | 1214.30 | 472.02 | 128.69 |
| size (μm) | | | | |
| Standard error | 322.41 | 211.87 | 116.60 | 22.87 |

### Experimental Example 3

### Analysis of Acid-Soluble Collagen According to the Extraction Time

To investigate the extraction of acid-soluble collagen according to the extraction time, the jellyfish pulverized in Example 1 was dipped in 0.5 M acetic acid (glacial grade, Merck, Darmstadt, Germany), which was irradiated with gamma-ray at the dosages of 10 kGy and 25 kGy, followed by stirring at 4° C. for 1, 3, and 5 days. Collagen was extracted by the same manner as described in Example 3. The obtained collagen was weighed, and the yield was calculated by the below mathematical formula 1 (FIG. 5).

Yield (%)=(weight of acid-soluble collagen extracted at x kGy/weight of acid-soluble collagen extracted at 0 kGy)×100 [Mathematical Formula 1]

As a result, as shown in FIG. 5, the collagen yield was increased as the number of days for stirring increased. For example, the yield after 3 or 5 day stirring was greater than the yield after 1 day stirring. As for the irradiation, the yield was increased when the jellyfish was irradiated with gamma-ray at the dosage of 25 kGy, compared with when the jellyfish was irradiated with gamma-ray at the dosage of 10 kGy (FIG. 5).

### Experimental Example 4

### Analysis of Acid-Soluble Collagen According to the Radiation Dose

To investigate the extraction yield of acid-soluble collagen according to the radiation dose, the jellyfish pulverized in Example 1 was dipped in 0.5 and 1 M acetic acid, followed by irradiation with gamma-ray at the dosages of 0, 10, 25, 50, and 100 kGy, followed by stirring at 4° C. for 2 weeks. Collagen was extracted by the same manner as described in Example 3. The obtained collagen was weighed, and the yield was calculated by the mathematical formula 1 (FIG. 6).

As a result, as shown in FIG. 6, the yield at the dosage of 0 kGy was considered as 100%. As the radiation dose increased, collagen yield increased. Particularly, the collagen yield was as significantly increased as 421.20±67.66% at the dosage of 25 kGy (FIG. 6).

### Experimental Example 5

### Analysis of Attelo Collagen According to the Radiation Dose

To investigate the extraction yield of attelo collagen according to the radiation dose, the jellyfish pulverized in Example 1 was dipped in 0.5 and 1 M acetic acid, followed by irradiation with gamma-ray at the dosages of 0, 10, 25, 50, and 100 kGy, followed by stirring at 4° C. for 2 weeks. The acid-soluble collagen was extracted by the same manner as described in Example 3. Then, the separated acid-soluble collagen was dipped in the mixed solution comprising 0.5 M acetic acid and 5 w/w % pepsin (EC 3.4.23.1, 2× crystallized, Tokyo chemical industry, Japan), followed by stirring at 4° C. for 24 hours. Attelo collagen was extracted by the same manner as described in Example 4. The obtained attelo collagen was weighed, and the yield was calculated by the below mathematical formula 2.

Yield (%)=(weight of attelo collagen after pepsin treatment/weight of acid-soluble collagen)×100 [Mathematical Formula 2]

As a result, as shown in FIG. 7, the weight of attelo collagen produced after the treatment of pepsin was reduced, but the weight change was less when attelo collagen was irradiated with gamma-ray at a high dosage. When collagen was extracted after being irradiated, the yield could be raised (FIG. 7).

### Experimental Example 6

### Investigation of Chemical Properties of Collagen According to the Radiation Dose

The chemical properties of the acid-soluble collagen was investigated by using ATR-FTIR spectrophotometer.

Particularly, the pulverized jellyfish prepared by the same manner as described in Experimental Example 4 was irradiated with gamma-ray at the dosages of 0, 10, and 25 kGy, followed by extraction of collagen. The extracted collagen and the animal originated collagen ‘rat tail type I collagen’ were analyzed by using ATR-FTIR spectrophotometer (Bruker TEMSOR 37, Bruker AXS. Inc., Germany). The analysis conditions were as follows; spectrum range: 500-4000 cm⁻¹, ATR mode, number of scanning: 64, and resolving power: 4 cm⁻¹.

As a result, the chemical properties of the acid-soluble collagen were confirmed as shown in FIG. 8. The marine organism originated collagen demonstrated the animal collagen like spectrum pattern. Amide A, I and II regions are directly related to the pattern of polypeptide. Amide A region (3400-3440 cm⁻¹) is related to N—H stretching and amide I region (1600-1660 cm⁻¹) is related to the stretching vibrations of carbonyl group and is useful for the investigation of the secondary structure of protein. Amide II region (−1550 cm⁻¹) is related to NH bending and CN stretching, and also related to the triple helical structure of collagen. Jellyfish collagen was identified with amide I, amide II, and amide A peaks respectively at 1635 cm⁻¹, 1530 cm⁻¹, and 3280 cm⁻¹ (FIG. 8).

### Experimental Example 7

### Investigation of Thermal Characteristics of Collagen According to the Radiation Dose

The chemical properties of the acid-soluble collagen was investigated by using differential scanning calorimeters.

Particularly, the pulverized jellyfish prepared by the same manner as described in Experimental Example 4 was irradiated with gamma-ray at the dosages of 0, 10, and 25 kGy, followed by extraction of collagen. The extracted collagen and rat tail type I collagen were analyzed by using differential scanning calorimeters (TA Q100, TA instruments, USA). The samples were measured in nitrogen environment at the temperature range of 0˜300° C. with the heating rate of 10° C./min.

As a result, as shown in FIG. 9, the acid-soluble collagen extracted after being irradiated with gamma-ray at the dosages of 10 kGy and 25 kGy showed similar pattern over the temperature change to the collagen extracted from jellyfish not-irradiated with gamma-ray. Particularly, the acid-soluble collagen extracted from jellyfish after being irradiated at the dosage of 10 kGy showed almost the same pattern as the collagen extracted from jellyfish not-irradiated with gamma-ray. Therefore, it was confirmed that the irradiation treatment for increasing collagen yield from jellyfish did not affect the thermal characteristics of collagen (FIG. 9).

### Experimental Example 8

### SDS-PAGE of Attelo Collagen According to the Radiation Dose

The changes of attelo collagen composition according to the radiation dose were analyzed by SDS-PAGE.

Particularly, electrophoresis was performed by using Mini-Protean 3 (Bio-Rad Laboratories, Hercules, Calif.) according to the method of Laemmli (1970). The polyacrylamide gel was prepared with stacking gel and resolving gel, 5% each. As explained in Experimental Example 5, jellyfish was dipped in 0.5 M acetic acid and irradiated with gamma-ray at the dosages of 0, 10, and 25 kGy, by which attelo collagen was extracted. The concentration of attelo collagen sample was adjusted with distilled water to 20 mg/ml, which was mixed with 0.25 M Tris-HCl (pH 6.8) containing 10% SDS, 20% glycerol, 5% 2-mercaptoethanol, and 0.1% bromophenol blue. The mixture was heated at 100° C. for 3 minutes. Rat tail type I collagen was prepared by the same manner as used for the preparation of the above jellyfish collagen and then used as the control protein for the comparative analysis. The prepared jellyfish collagen sample and rat tail type I collagen sample were loaded on polyacrylamide gel, followed by electrophoresis at 20 mA/gel. Upon completion of electrophoresis, the gel was stained with 0.25% (w/v) Coomassie brilliant blue R250, followed by de-coloring with methanol/acetic acid mixture. Then, the jellyfish collagen and rat tail type I collagen were compared.

As a result, as shown in FIG. 10, the rat tail type I collagen was composed of two α1-chains, α2-chain and β-component (α-chain cross-linked dimer). In the meantime, α1-chain and α2-chain of the jellyfish originated collagen displayed same level of mobility but β-component was weak (FIG. 10).

### Experimental Example 9

### Analysis of Attelo Collagen Amino Acid According to the Radiation Dose

Amino acids of the attelo collagen extracted from jellyfish that had been dipped in 0.5 M acetic acid and irradiated with gamma ray by the same manner as described in Experimental Example 5 and the rat tail type I collagen were analyzed.

As a result, as shown in Table 3, both jellyfish collagen and rat tail type I collagen had plenty of glycine, alanine, and proline, which is typical characteristics of collagen. In particular, the amino acid composition of the attelo collagen that had not been irradiated with gamma-ray was not different from that of the attelo collagen irradiated with gamma-ray at the dose of 10 kGy. The content of hydroxyproline in the jellyfish collagen of the present invention was lower than that in the mammal originated rat rail type I collagen (Table 3).

TABLE 3

| | | |
|---|---|---|
| | Attelo collagen | |
| | (Radiation dose, | Rat tail |
| Amino acid | kGy) | type I |

| (unit: mol %) | 0 | 10 | collagen |
|---|---|---|---|
| Cysteine and cystine | 0.75 | 0.64 | 0.16 |
| Asparagines and aspartic | 2.25 | 3.04 | 4.70 |
| acid | | | |
| Glutamine and glutamic | 9.06 | 9.30 | 7.57 |
| acid | | | |
| Hydroxyproline | 0.40 | 1.08 | 9.38 |
| Serine | 6.00 | 6.23 | 3.20 |
| Glycine | 10.05 | 10.62 | 32.90 |
| Histidine | 1.14 | 1.07 | 0.43 |
| Arginine | 3.37 | 3.96 | 5.29 |
| Threonine | 6.67 | 6.24 | 1.80 |
| Alanine | 9.73 | 10.02 | 11.48 |
| Proline | 12.21 | 9.42 | 12.80 |
| Tyrosine | 2.83 | 2.12 | 0.19 |
| Valine | 6.88 | 8.14 | 2.41 |
| Methionine | 2.03 | 1.30 | 0.59 |
| Isoleucine | 6.44 | 7.29 | 1.23 |
| Leucine | 8.66 | 8.90 | 2.32 |
| Phenylalanine | 4.68 | 3.17 | 1.25 |
| Tryptophan | 1.46 | 0.68 | 0.17 |
| Lysine | 5.10 | 6.78 | 2.12 |
| Total | 100 (%) | 100 (%) | 100 (%) |

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

1. A method for separating acid-soluble collagen from jellyfish comprising the following steps:
1) washing and pulverizing jellyfish;
2) dipping the pulverized jellyfish prepared in step 1) in an acid solution;
3) irradiating the solution of step 2), followed by stirring; and
4) filtering the stirred solution of step 3) and drying thereof.

2. The method for separating acid-soluble collagen from jellyfish according to claim 1, wherein the step 1) is performed by the following steps:
I) pulverizing the washed jellyfish;
II) freeze-drying the pulverized jellyfish of step I); and
III) pulverizing the freeze-dried jellyfish of step II).

3. The method for separating acid-soluble collagen from jellyfish according to claim 1, wherein the freeze-dried jellyfish is pulverized in the particle size of 100˜3000 μm in step III).

4. The method for separating acid-soluble collagen from jellyfish according to claim 1, wherein the acid solution of step 2) is selected from the group consisting of acetic acid solution, citric acid solution, and formic acid solution.

5. The method for separating acid-soluble collagen from jellyfish according to claim 1, wherein the concentration of the acid solution of step 2) is 0.01 M-2.0 M.

6. The method for separating acid-soluble collagen from jellyfish according to claim 1, wherein the radiation of step 3) is gamma-ray or electric beam.

7. The method for separating acid-soluble collagen from jellyfish according to claim 1, wherein the irradiation of step 3) is performed at the dose of 5 kGy˜200 kGy.

8. The method for separating acid-soluble collagen from jellyfish according to claim 1, wherein the step 4) is performed by the following steps:
i) obtaining the precipitate from the filtrate remaining after filtering the stirred solution;
ii) obtaining the supernatant after dissolving the precipitate of step i) in an acid solution;
iii) obtaining the precipitate by adding salt to the supernatant of step ii); and
iv) dissolving the precipitate of step iii) in an acid solution, followed by dilution and freeze-drying.

9. A method for preparing attelo collagen containing the step of treating the acid-soluble collagen prepared by the methods of claim 1 with protease and drying the resultant product.

10. The method for preparing attelo collagen according to claim 9, wherein the protease is pepsin or trypsin.

11. The method for preparing attelo collagen according to claim 9, wherein the protease is added at the concentration of 1˜10 (w/w) %.

12. The method for preparing attelo collagen according to claim 9, wherein the protease is functioning to eliminate telo peptide of collagen.

13. The method for preparing attelo collagen according to claim 9, wherein the drying is performed by quick freezing at −178˜−70° C.

14. The method for preparing attelo collagen according to claim 9, wherein the method is composed of the following steps:
a) dissolving the acid-soluble collagen in the acid/pepsin mixed solution, followed by stirring;
b) dissolving the precipitate obtained from the stirred mixture of step a) in acid, to which salt is added to precipitate collagen; and
c) dissolving the precipitated collagen of step b) in acid, followed by dilution and freezing.
